(19) United States
(12) Patent Application Publication
(10) Pub. No.: US 2026/0216449 A1
(43) Pub. Date: Jul. 30, 2026

(54) A SUBASSEMBLY OF A MEDICAMENT DELIVERY DEVICE

(71) Applicant: SHL Medical AG, Zug (CH)

(72) Inventors: Johan Zander, Nacka Strand (SE); Johan Egerström, Nacka Strand (SE)

(21) Appl. No.: 19/143,029

(22) PCT Filed: Dec. 20, 2023
(86) PCT No.: PCT/EP2023/086832

§ 371 Date: Jun. 25, 2025

(30) Foreign Application Priority Data
Jan. 3, 2023 (EP) 23150105.7

(51) Int. Cl. A61M 5/32

(52) U.S. Cl. CPC A61M 5/3257; A61M 2005/3247; A61M 2005/3258

(57) ABSTRACT

The present disclosure generally relates to medicament delivery devices such as autoinjectors, and particularly concerns a subassembly comprising separate springs for activation and lock out after use.

### TECHNICAL FIELD

The present disclosure generally relates to medicament delivery devices such as autoinjectors, and particularly concerns a subassembly of a medicament delivery device.

### BACKGROUND

A number of medical conditions require injections. These days, a number of different injection devices exist, including various types of pen injectors, autoinjectors and on-body devices. Although many of these devices have enabled major improvements in the management of a number of medical conditions, various limitations do still exist in the current technology. Not least amongst these are the difficulties faced by patients that require frequent injections and by patients that need to inject particularly viscous drugs. In considering these problems, the applicant has appreciated that various developments could be made to help improve the medicament delivery devices on the market today, for example concerning activation of the medicament delivery devices into a soft tissue, which are set out in more detail below.

### SUMMARY

An object of the present disclosure is to provide a subassembly for a medicament delivery device which solves, or at least mitigates problems of the prior art.

According to a first aspect of the present disclosure, there is provided a subassembly of a medicament delivery device, the subassembly comprising: a housing extending in a longitudinal direction from a proximal end to a distal end relative a longitudinal axis, a needle cover arranged in the housing and configured to cover a needle at the proximal end of the housing, the needle cover is axially movable in relation to the housing along the longitudinal axis between a distal position and a proximal position, a rotator arranged in the needle cover, the rotator is axially rotatable with respect to the needle cover, the rotator comprises a lock member with a distally facing surface that abuts against a proximally facing surface of a corresponding lock portion of the needle cover, to prevent the needle cover from moving linearly in the proximal direction in relation to the rotator, a first spring arranged in the rotator, the first spring is compressed between a proximally facing surface of the rotator and a distally facing surface of the needle cover, and a second spring arranged in the housing, the second spring is arranged between a proximally facing surface of the housing and a distally facing surface of the rotator, wherein the stiffness of the first spring is higher than the stiffness of the second spring.

Embodiments of the present disclosure advantageously provides for a subassembly that improves the ability to activate a medicament delivery device into soft tissue. Typically, activation into soft tissue may be difficult due to the relatively high activation force employed in prior art devices. The high activation force is often related to that a high lock-out force is required after injection to prevent sharp needle injuries. However, this causes problems when attempting to active the device against a soft tissue of an obese patient since the force applied by the device deform the soft tissue instead of activating the device. Herein, the inventors propose a dual-spring solution where a first spring is compressed between a proximally facing surface of the rotator and a distally facing surface of the needle cover, and may provide a lock-out function, and a second spring arranged between a proximally facing surface of the housing and a distally facing surface of the rotator and may provide a device activation force. Advantageously, the stiffness of the first spring is higher than the stiffness of the second spring. An advantage that this solution can provide is that the activation force of a medicament delivery device and the medicament delivery member guard lock-out force are decoupled and can be set independently by varying the strengths of the two springs.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the components thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially” refer to a circumference or a circumferential direction 301 relative to an axis 102, typically a central axis extending in the direction of the longest extension of the device and/or component. Similarly, “radial” or “radially” refer to a direction 302 extending radially relative to the axis, and “rotation”, “rotational” and “rotationally” refer to rotation relative to the axis.

According to one embodiment, the abutting between the distally facing surface of the lock member and the proximally facing surface of the corresponding lock portion may prevent linear motion of the needle cover in the proximal direction in relation to the rotator during an initial rotational motion of the rotator relative the needle cover guided by the lock portion. The initial rotation may be part of an activation stroke where the second spring is compressed. During this initial rotation, the needle cover is advantageously maintained linearly locked in relation to the rotator.

According to one embodiment, the lock member may be a radially extending protrusion of the rotator and the lock portion is a slot in the needle cover. This slot and protrusion arrangement provides a reliable and robust combined guiding and locking solution.

According to one embodiment, the slot may comprise a first portion that is curved along a circumference of the needle cover, and a second portion that extend parallel with the longitudinal axis. The first portion comprises the proximally facing surface of the lock portion. The first portion allows only for a rotation of the rotator with respect to the needle cover whereas the second portion only allows for a linear motion of the rotator with respect to the needle cover and vice versa.

According to one embodiment, the rotator may comprise a distally facing surface arranged facing a proximally facing surface of the housing, wherein one of the distally facing surface of the rotator and the proximally facing surface of the housing is inclined with respect to the longitudinal axis such that the rotator rotates through an initial rotation angle about the longitudinal axis when the distally facing surface of the rotator is axially moved in engagement to slide against the proximally facing surface of the housing. The inclined surface provides an efficient way to transfer a linear motion to a rotational motion.

According to one embodiment, the lock member may be engaged with the corresponding lock portion of the needle cover to prevent the needle cover from moving linearly in the proximal direction in relation to the rotator throughout the rotation of the rotator through the initial rotation angle.

According to one embodiment, the rotator may comprise a second set of radially extending protrusions that are longitudinally aligned with a second distally facing surface of the housing after the rotation of the rotator through the initial rotation angle.

According to one embodiment, one of the second set of radially extending protrusions and the second distally facing surface of the housing may comprise a cam surface that is inclined with respect to the longitudinal axis, wherein the second spring is configured to, subsequent to an injection event, decompress to linearly move the second set of radially extending protrusions and the second distally facing surface of the housing in engagement whereby the rotator rotates through a further rotation angle about the longitudinal axis. Thus, the cam surfaces provide an efficient way to transfer a linear motion to a rotational motion to here rotate the rotator further to prepare the rotator for a further action.

According to one embodiment, the further rotation angle may be sufficient to move the lock member to a second portion of the lock portion which allows linear motion between the rotator and the needle cover. Hereby, the first spring advantageously push the needle cover proximally to shield the needle after the injection event.

According to one embodiment, the locking member may be an l-shape slot.

According to one embodiment, the first spring and the second spring may be coaxially arranged. This advantageously aligns the spring forces of the two springs.

According to one embodiment, the stiffness of first spring may be substantially higher than the stiffness of the second spring. For example, the spring force provided by first spring may be at least twice that of the second spring.

According to one embodiment, the second spring may be configured as an activation spring that is compressed during an injection event. To allow for activation into soft tissue, the stiffness of this spring should be relatively low.

According to one embodiment, the first spring may be a lock-out spring for a lock-out mechanism configured to shield the needle with the needle cover subsequent to an injection event. To allow reliable protection of the needle after an activation event, the stiffness of the first spring should be relatively high.

There is further provided a medicament delivery device comprising the subassembly of any of the herein disclosed embodiments.

In summary, the present disclosure generally relates to medicament delivery devices such as autoinjectors, and particularly concerns a subassembly comprising separate springs for activation and lock out after use.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of an autoinjector according to embodiments of the present disclosure;

FIG. 2 is an exploded view of a subassembly according to embodiments of the present disclosure;

FIG. 3 is a perspective view of a rotator according to embodiments of the present disclosure;

FIG. 4 is a perspective view of a rotator according to embodiments of the present disclosure;

FIG. 5 is a perspective view of a rotator according to embodiments of the present disclosure;

FIG. 6 is a perspective view of a needle cover according to embodiments of the present disclosure;

FIG. 7 is a perspective view of a needle cover according to embodiments of the present disclosure;

FIG. 8 is a perspective view of a housing according to embodiments of the present disclosure;

FIG. 9A is a perspective view of a subassembly in an initial state according to embodiments of the present disclosure;

FIG. 9B is a perspective view of a subassembly without a housing in an initial state according to embodiments of the present disclosure;

FIG. 10A is a perspective view of a subassembly with the needle cover retracted according to embodiments of the present disclosure;

FIG. 10B is a perspective view of a subassembly illustrating an initial rotation of the rotator according to embodiments of the present disclosure;

FIG. 10C is a perspective view of a subassembly illustrating an initial rotation of the rotator according to embodiments of the present disclosure;

FIG. 10D is a perspective view of a subassembly illustrating an initial rotation of the rotator where a protrusion travels in a slot of the needle cover according to embodiments of the present disclosure;

FIG. 11A is a perspective view of a subassembly illustrating a further rotation of the rotator according to embodiments of the present disclosure;

FIG. 11B is a perspective view of a subassembly illustrating a further rotation of the rotator where the protrusion travels in the slot of the needle cover according to embodiments of the present disclosure;

FIG. 12A is a perspective view of a subassembly illustrating the protrusion travelling linearly in a slot of the needle cover according to embodiments of the present disclosure;

FIG. 12B is a perspective view of a subassembly with the needle cover in a lock-out position according to embodiments of the present disclosure; and

FIG. 12C is a perspective view of a subassembly with the needle cover in a lock-out position according to embodiments of the present disclosure;

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

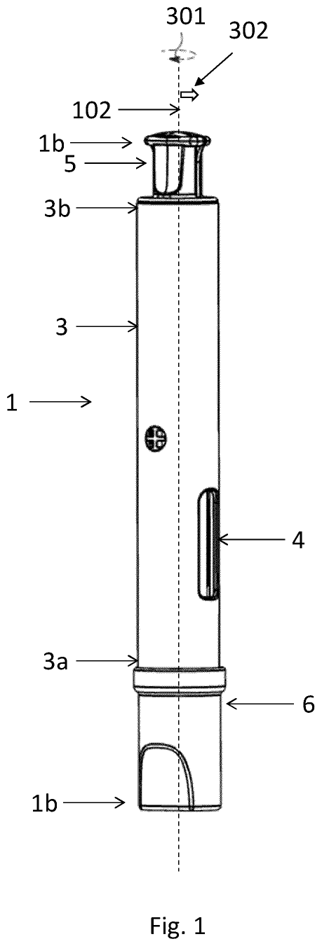

FIG. 1 shows an example of a medicament delivery device 1 such as an autoinjector according to embodiments of the present disclosure. The medicament delivery device 1 is configured to expel medicament from a medicament container, via a medicament delivery member such as a needle, to a user at a dose delivery site. The medicament delivery device 1 extends from a proximal end 1a to a distal end 1b relative to the axis 102.

The medicament delivery device 1 comprises a housing 3 with a window 4. The housing 3 has a proximal end 3a and a distal end 3b. A cap 6 covers the proximal end 3a of the housing 3 and a rear knob 5 is arranged at the distal end 3b.

The medicament delivery device 1 comprises a subassembly 2 which will now be described in more detail with reference to subsequent drawings.

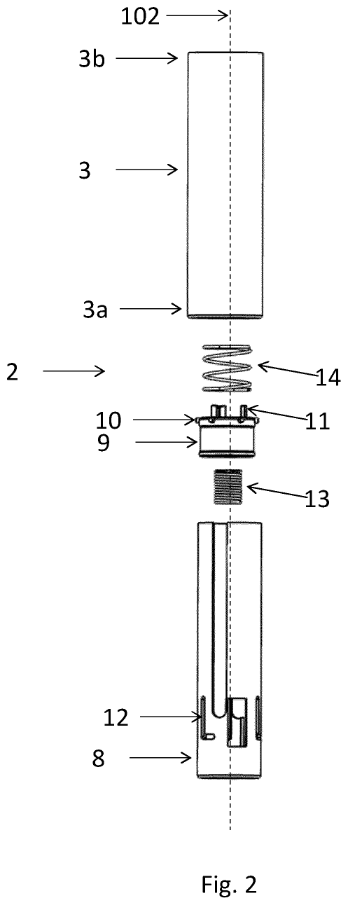

An exploded view of the subassembly 2 is shown in FIG. 2. The subassembly 2 comprises the housing 3 extending in a longitudinal direction from a proximal end 3a to a distal end 3b relative a longitudinal axis 102.

The subassembly 2 further comprises a needle cover 8 arranged in the housing 3 and configured to cover a needle at the proximal end 3a of the housing 3. The needle cover 8 is axially movable in relation to the housing 3 along the longitudinal axis 102 between a distal position and a proximal position. Generally, the needle cover 8 is configured to be moved linearly relative to the housing 3 from an extended position to a retracted position in which the needle cover 8 is received further in the housing 3 and in which the needle is exposed.

The subassembly comprises a rotator 9 arranged in the needle cover 8. The rotator is axially rotatable with respect to the needle cover 8. The rotator 9 comprises a lock member 10 and a distally extending protrusion 11. Correspondingly, the needle cover 8 comprises a lock portion 12.

The subassembly 2 further comprises a first spring 13 arranged in the rotator 9. The first spring 13 is compressed between a proximally facing surface of the rotator 9 and a distally facing surface of the needle cover 8.

The subassembly 2 further comprises a second spring 14 arranged in the housing. The second spring 14 is arranged between a proximally facing surface of the housing 3 and a distally facing surface of the rotator 9.

Advantageously, the stiffness of the first spring 13 is higher than the stiffness of the second spring 14. In some embodiments, the stiffness of the first spring 13 is substantially higher than the stiffness of the second spring 14. Furthermore, the first spring 13 and the second spring 14 are coaxially arranged, that is, they are arranged to share the longitudinal axis 102.

Different stiffness of springs can be achieved in various ways such as by material selection, the thickness of the coil, the number of turns per unit length of the spring, etc. As an example, the stiffness of the first spring may be two or more times greater than the stiffness of the second spring, for example two times the stiffness of the second spring.

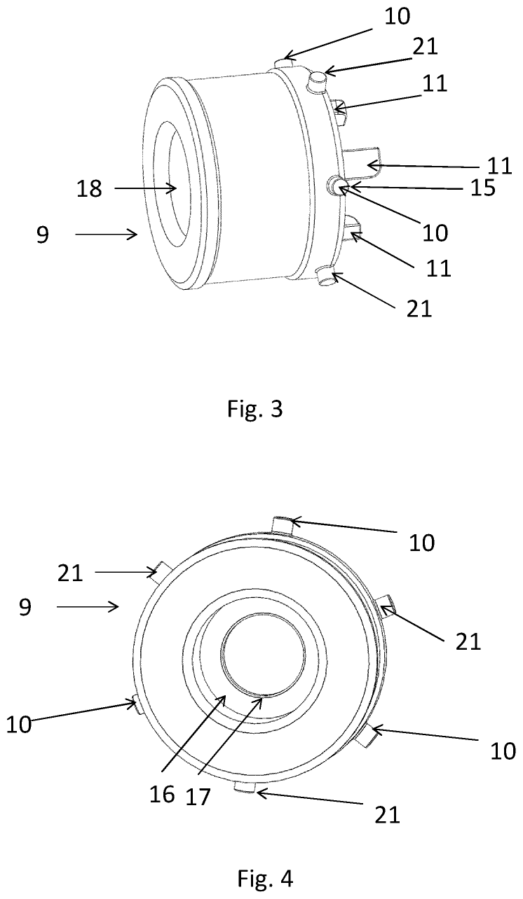

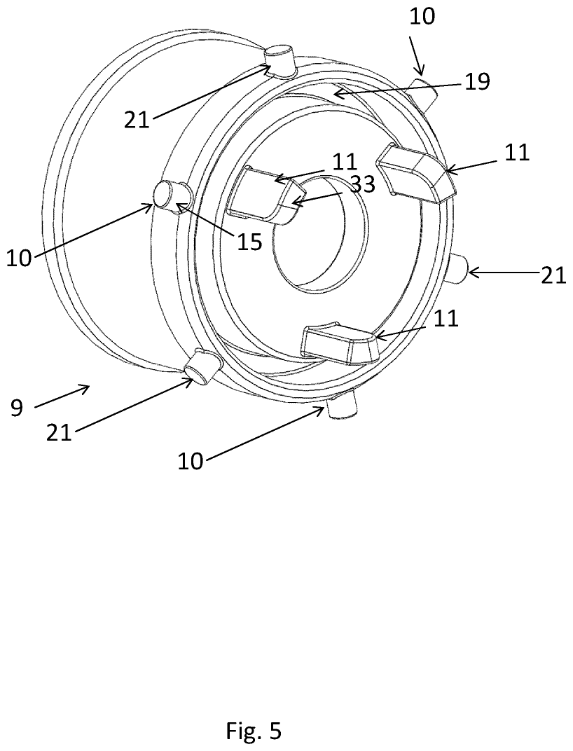

FIGS. 3-5 illustrate different perspective views of the rotator 9. The rotator 9 comprises lock members 10 annularly arranged about the circumference of the generally cylindrical rotator 9. The lock members 10 are equally distributed about the circumference. Here, the lock members 10 are radially extending protrusions of the rotator 9.

The protrusions 10 each comprise a distally facing surface 15.

Furthermore, the rotator 9 comprises distally extending protrusions 11, each comprising a distally facing surface 33 facing into the housing 3.

The first spring 13 is compressed between a proximally facing surface 16 of the rotator 9 and a distally facing surface of the needle cover 8. The proximally facing surface 16 is comprised in an inner annular flange 17 of the rotator 9. The rotator 9 further comprises an axial through-hole 18.

In addition, the rotator 9 comprises a second set of radially extending protrusions 21 that are annularly arranged about the circumference of the rotator 9. The second set of radially extending protrusions 21 are interleaved with the protrusions 10.

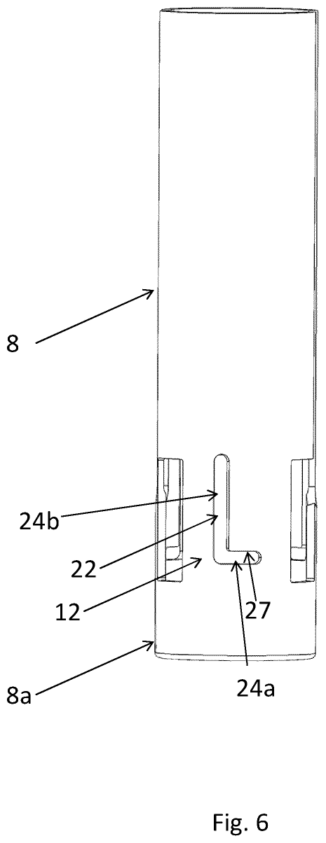

Turning specifically to FIG. 5, the rotator 9 comprises a distally facing surface 19 on which the second spring 14 is supported. The second spring 14 is arranged between a proximally facing surface of the housing 3 and the distally facing surface 19 of the rotator 9. The distally facing surface 19 is located in an annular trench 20 of the rotator 9 with a diameter that matches that of the second spring 14. Thus, the second spring 14 can be fitted partly into the trench 20 with a distal part of the second spring 14 extending out from the trench 20 to reach the proximally facing surface of the housing 3.

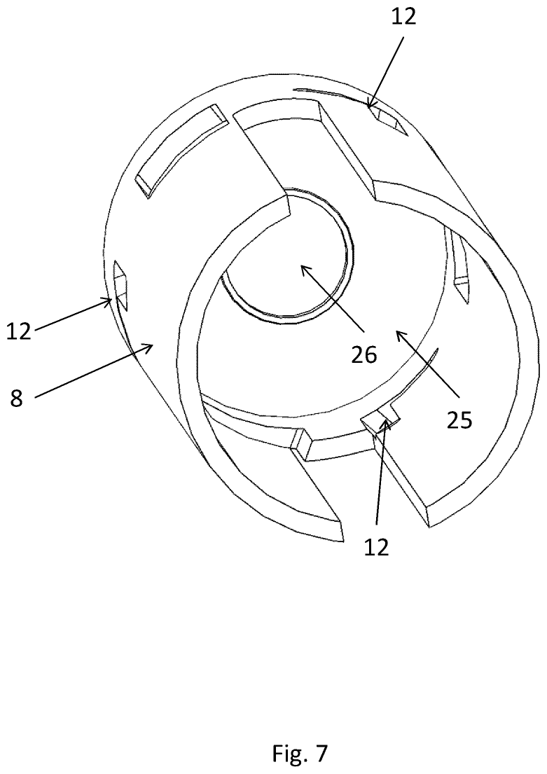

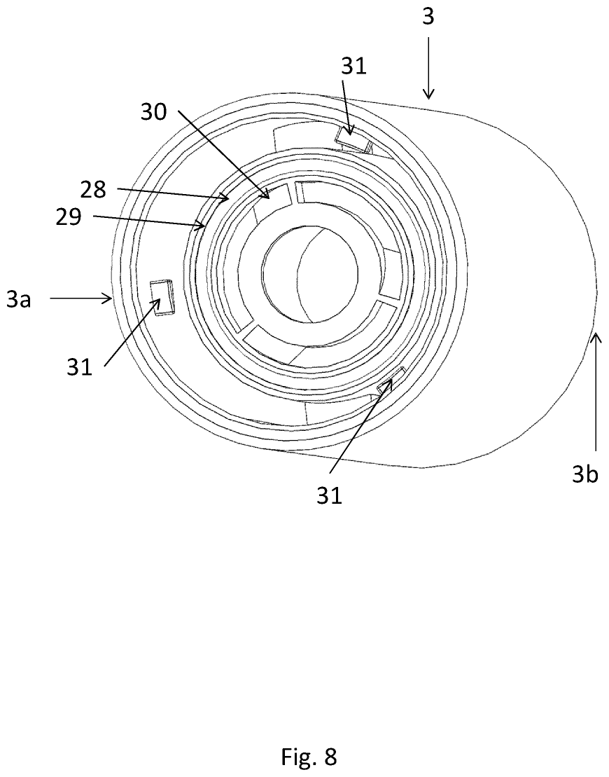

FIGS. 6 and 7 are different perspective views of the needle cover 8. The needle cover 8 comprises a lock portion 12. In this specific embodiment, the needle cover 8 comprises three lock portions 12 annularly arranged about the circumference of the needle cover 8 at locations to match the annularly arranged protrusions 10 of the rotator 9.

The lock portion 12 is a slot 22 in the needle cover 8. The slot 22 comprises a first portion 24a that is curved along a circumference of the needle cover 8, and a second portion 24b that extend parallel with the longitudinal axis 102. The first portion 24a and the second portion 24 b meet at an angle which may be about 90 degrees. The slot 22 may be an L-shaped slot. The slot 22 comprises a proximally facing surface 27, here being part of the first slot portion 24a.

Turning specifically to FIG. 7, the needle cover comprises a distally facing surface 25. The distally facing surface 25 is at the proximal end 8a of the needle cover 8, and the distally facing surface 25 faces the rotator 9. The distally facing surface 25 surrounds an opening 26 where a needle protrudes during an injection event. The first spring 13 is compressed between the proximally facing surface 16 of the rotator 9 and the distally facing surface 25 of the needle cover 8.

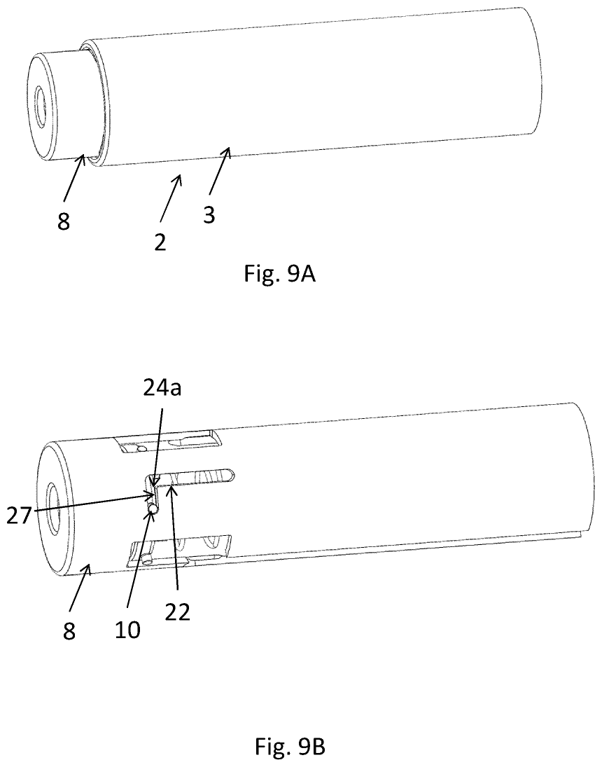

FIG. 8 is a perspective view of the housing from the proximal end 3a. Inside the housing 3, there is a proximally facing surface 28. The proximally facing surface 28 is annular and arranged in a trench 29 of the housing 3. The diameter of the trench 29 matches that of the second spring 14 so that a distal end of the second spring 14 is arranged in the trench. The second spring 14 is arranged between the proximally facing surface 28 of the housing 3 and the distally facing surface 19 of the rotator 9. Furthermore, the housing 8 comprises a proximally facing surface 30 that faces the distally facing surface 33 of the rotator 9. The housing 3 also comprises second distally facing surface formed on radially inwards extending protrusions 31 formed on the inside of the wall of the housing 3.

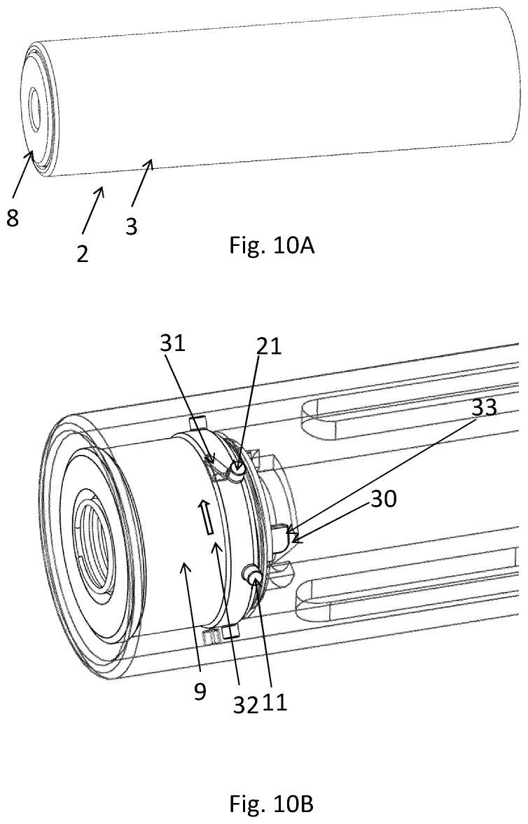

Turning to FIGS. 9A-B where the subassembly 2 is shown in an initial state. In FIG. 9A, the needle cover 8 extends proximally outside the housing 3. FIG. 9B shows the subassembly in FIG. 9A but with the housing 3 removed. The protrusion 10 of the rotator 9 is arranged in the slot 24a of the lock portion 12 in the needle cover 8. With the second spring being compressed between the rotator and the needle cover, the second spring attempts to push the needle cover 8 proximally, however the abutting contact between the protrusion 10 and the proximally facing surface 27 of the slot 24a prevents such linear motion at least in the proximal direction.

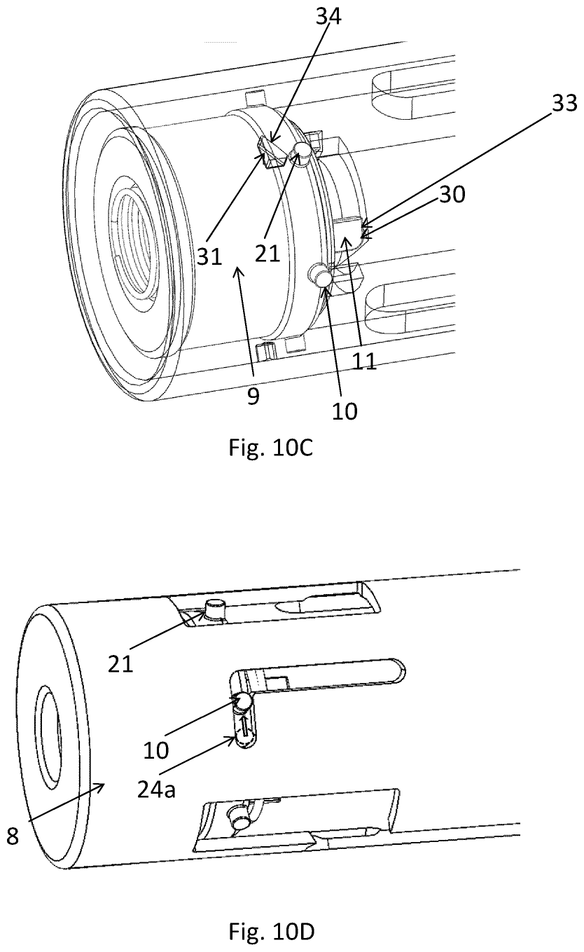

FIG. 10A the subassembly 2 is shown with the needle cover 8 in a retracted position where the needle cover 8 is received further in the housing 3. In this position, the distally facing surface 33 of the rotator 9 abuts against the proximally facing surface 30 of the housing. Furthermore, the second spring 14 is compressed between the proximally facing surface 28 of the housing 3 and the distally facing surface 19 of the rotator 9.

The second spring 14 is configured as an activation spring that is compressed during an injection event. The stiffness of the second spring 14 is relatively low, or soft, to facilitate activation, especially for soft tissue patients that may suffer from obesity. In other words, the activation force for initiating an injection is relatively low.

In this embodiment, the proximally facing surface 30 of the housing 3 is inclined with respect to the longitudinal axis 102 such that the rotator 9 rotates through an initial rotation angle about the longitudinal axis 102 when the distally facing surface 33 of the rotator 9 is axially moved in engagement to slide against the proximally facing surface 30 of the housing. Equally well, the inclined surface may be part of the distally extending protrusion 11 instead of the housing 3.

The interaction between the proximally facing surface 30 of the housing 3 and the distally facing surface 33 of the rotator 9 cause the rotator to rotate as indicated by arrow 32 through an initial rotation angle to a second position shown in FIG. 10C.

The rotation of the rotator 9 causes the protrusion 10 to travel along the first slot portion 24a as shown in FIG. 10D. However, the travel distance of the protrusion 10 is shorter than the length of the first slot portion 24a so that engagement between the slot portion 24a and the protrusion 10 still prevents proximal linear motion of the needle cover 8 in relation to the rotator during the initial rotational motion, through the initial rotation angle, of the rotator relative the needle cover 8. The rotation may be guided by the locking portion 12, i.e., here more specifically guided by the first slot portion 24a.

As a result of the initial rotation of the rotator 9, the second set of radially extending protrusions 21 are longitudinally aligned with a second distally facing surface 34 of the housing 3. Thus, the second set of protrusions are moved from being misaligned with the radially inwards extending protrusions 31 to being located directly distally from the radially inwards extending protrusions 31 each comprising a second distally facing surface 34.

Once injection is performed and the needle cover 8 is removed from an injection site, the second spring 14 push the needle cover 8 in the proximal direction.

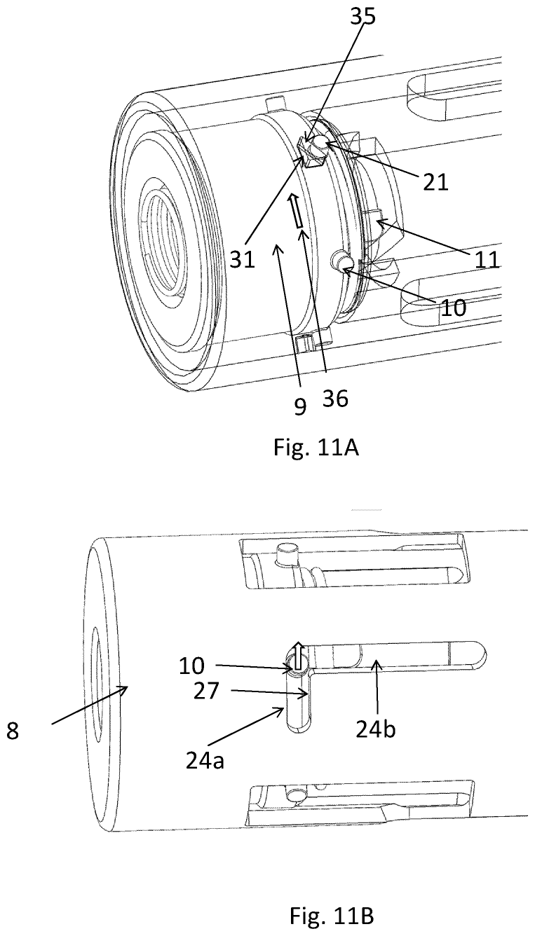

Turning to FIG. 11A, the second distally facing surface 34 of the housing comprises a cam surface 35 that is inclined with respect to the longitudinal axis 102, although it is equally applicable that the cam surface is part of the protrusion 21. When the second spring decompresses subsequent to an injection event, the second set of radially extending protrusions 21 are moved to engage with the second distally facing surfaces 35 of the housing 3. The interaction between the cam surface 35 and a proximally facing surface of the protrusion 21 causes the rotator 9 to rotate through a further rotation angle about the longitudinal axis 102, as indicated by arrow 36. Thus, the second spring 13 pushes the rotator proximally so that the protrusion 21 engages with the cam surface 35, whereby the rotator 9 rotates further in the same direction as during the initial rotation.

Turning to FIG. 11b, the subassembly 2 is again shown without the housing. During this further rotation, the protrusion 10 moves further in the first slot portion 24a to reach the second slot portion 24b which is linear and parallel with the longitudinal axis 102. The further rotation angle is sufficient to move the protrusion 10 to the second portion 24b of the lock portion 12 which allows linear motion between the rotator 9 and the needle cover 8. In other words, the locking abutting between the proximally facing surface 27 of the first slot portion 24a and the protrusion 10 is lost and the needle cover 8 can move linearly with respect to the rotator 9 and the housing.

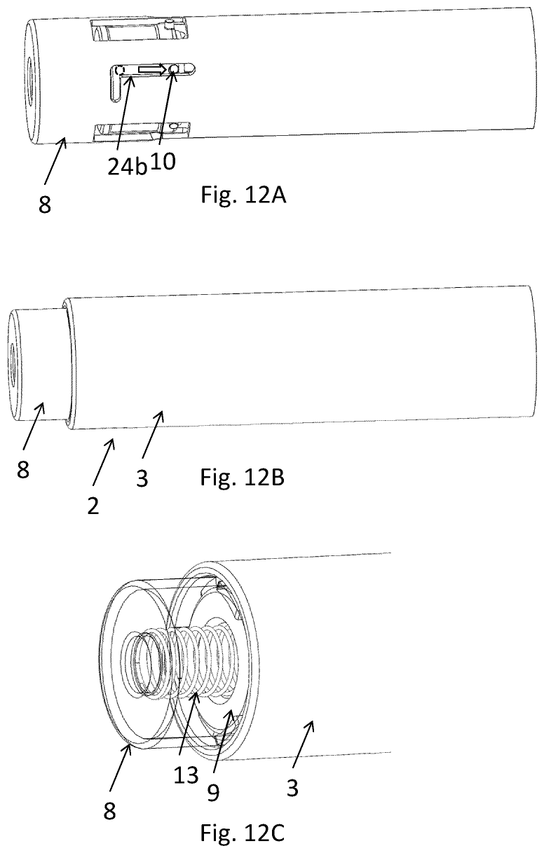

Turning to FIG. 12A, once the protrusion 10 reaches the second slot portion 24b, the protrusion 10 travels along the slot portion 24b as the first spring 13 is decompressed. Thus, under the spring force of the first spring 13, the needle cover 8 travels proximally which causes the protrusion 10 to move relative the second slot portion 24b.

Turning to FIG. 12B, the subassembly 2 is illustrated in a lock-out state subsequent to an injection event. The needle cover 8 is now in the extended position where it shields a needle to prevent sharp injury.

FIG. 12C illustrates the first spring 13 compressed between the needle cover 8 and the rotator 9 subsequent to the injection event. The spring force of the first spring 13 is relatively high to provide a lock-out function. More specifically, the first spring 13 is a lock-out spring for a lock-out mechanism configured to shield the needle with the needle cover subsequent to an injection event.

A medicament delivery device (such as an autoinjector) may generally include various other components. For example, a sensor unit which may recognize injection events, such as the autoinjector inserted into an attachment portion of e.g., a pad, injection started, and injection ends, a memory unit which is configured to store the recorded data during the injection, a connectivity unit configured to transmit the stored data to a smart device or the network directly, a processing unit configured to control the entire system and processes the data before transmitting it, and/or user interface units that are configured to provide feedback to the patient, such as status LEDs, haptic, and/or audio feedback.

When the medicament delivery device is placed into the attachment portion, the sensors inside of the support pad are configured to recognize the event and give feedback to the patient via haptic/visual or audio elements.

When the injection finishes, the sensors are configured to recognize the event and give feedback to the patient again. Further, the collected data is stored in the memory unit and may be transmitted to the smart device/network via the connectivity unit after the injection event finishes.

The sensor can be one of or the combination of the following: a mechanical switch, a Hall-effect sensor, an accelerometer.

The mechanical switch, hall-effect sensor, or accelerometer can be used for detection of the insertion of the auto-injector into an injection port.

The accelerometer can be used for detecting injection events.

Possible wireless communication methods include Bluetooth and Cellular Networks.

Bluetooth connectivity requires a smart device to transmit the stored data to the network and it requires a pairing action between the support pad and the smart device before being able to use the supporting pad. But it's a cheaper alternative and it requires less space on PCB.

The cellular network does not require any pairing process, it can be used as a plug-n-play device, no prior setup is needed, but it's more expensive and it requires more space on PCB.

Depending on the requirements of the product any of those two technologies can be used.

Such processing units may comprise a logic circuit or control unit including a microprocessor, microcontroller, programmable digital signal processor or another programmable device. The processing circuitry may also, or instead, each include an application specific integrated circuit, a programmable gate array or programmable array logic, a programmable logic device, or a digital signal processor. Where the processing circuitry includes a programmable device such as the microprocessor, microcontroller or programmable digital signal processor mentioned above, the processor may further include computer executable code that controls operation of the programmable device.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47(CD47) antagonists, cluster of differentiation 30 (CD30) modulators, cluster of differentiation 73 (CD73 ) modulators, cluster of differentiation 66 (CD66) modulators, cluster of differentiation w137 (CDW137) agonists, cluster of differentiation 158 (CD158) modulators, cluster of differentiation 27 (CD27) modulators, cluster of differentiation 58 (CD58 ) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDW123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine. Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOpAD, 7+3, 5+2 , 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

The inventive concept has mainly been described above with reference to a few examples. However, as is readily appreciated by a person skilled in the art, other embodiments than the ones disclosed above are equally possible within the scope of the inventive concept, as defined by the appended claims.

1-15. (canceled)

16. A subassembly of a medicament delivery device, the subassembly comprising:
a housing extending in a longitudinal direction from a proximal end to a distal end relative a longitudinal axis;
a needle cover arranged in the housing and configured to cover a needle at the proximal end of the housing, the needle cover is axially movable in relation to the housing along the longitudinal axis between a distal position and a proximal position;
a rotator arranged in the needle cover, the rotator is axially rotatable with respect to the needle cover, the rotator comprises a lock member with a distally facing surface that abuts against a proximally facing surface of a corresponding lock portion of the needle cover, to prevent the needle cover from moving linearly in the proximal direction in relation to the rotator;
a first spring arranged in the rotator, the first spring is compressed between a proximally facing surface of the rotator and a distally facing surface of the needle cover; and
a second spring arranged in the housing, the second spring is arranged between a proximally facing surface of the housing and a distally facing surface of the rotator, wherein the stiffness of the first spring is higher than the stiffness of the second spring.

17. The subassembly of claim 16, wherein the abutment between the distally facing surface of the lock member and the proximally facing surface of the corresponding lock portion prevents linear motion of the needle cover in the proximal direction in relation to the rotator during an initial rotational motion of the rotator relative the needle cover guided by the locking portion.

18. The subassembly of claim 16, wherein the lock member is a radially extending protrusion of the rotator and the lock portion is a slot in the needle cover.

19. The subassembly of claim 18, wherein the slot comprises a first portion that is curved along a circumference of the needle cover, and a second portion that extend along the longitudinal axis.

20. The subassembly of claim 16, wherein the rotator comprises a distally facing surface arranged facing a proximally facing surface of the housing, wherein one of the distally facing surface of the rotator and the proximally facing surface of the housing is inclined with respect to the longitudinal axis such that the rotator rotates through an initial rotation angle about the longitudinal axis when the distally facing surface of the rotator is axially moved in engagement to slide against the proximally facing surface of the housing.

21. The subassembly of claim 20, wherein the lock member is engaged with the corresponding lock portion of the needle cover to prevent the needle cover from moving linearly in the proximal direction in relation to the rotator throughout the rotation of the rotator through the initial rotation angle.

22. The subassembly of claim 20, wherein the rotator comprises a second set of radially extending protrusions that are longitudinally aligned with a second distally facing surface of the housing after the rotation of the rotator through the initial rotation angle.

23. The subassembly of claim 22, wherein one of the second set of radially extending protrusions and the second distally facing surface of the housing comprises a cam surface that is inclined with respect to the longitudinal axis, wherein the second spring is configured to, subsequent to an injection event, decompress to linearly move the second set of radially extending protrusions and the second distally facing surface of the housing in engagement whereby the rotator rotates through a further rotation angle about the longitudinal axis.

24. The subassembly of claim 23, wherein the further rotation angle is sufficient to move the lock member to a second portion of the lock portion which allows linear motion between the rotator and the needle cover.

25. The subassembly of claim 16, wherein the locking member is an L-shape slot.

26. The subassembly of claim 16, wherein the first spring and the second spring are coaxially arranged.

27. The subassembly of claim 16, stiffness of first spring is substantially higher than the stiffness of the second spring.

28. The subassembly of claim 16, wherein the second spring is configured as an activation spring that is compressed during an injection event.

29. The subassembly of claim 16, wherein the first spring is a lock-out spring for a lock-out mechanism configured to shield the needle with the needle cover subsequent to an injection event.

30. A medicament delivery device comprising the subassembly of claim 16.
